(19) United States
(12) Patent Application Publication
(10) Pub. No.: US 2025/0082892 A1
(43) Pub. Date: Mar. 13, 2025

(54) INFORMATION PROCESSING DEVICE, INFORMATION PROCESSING METHOD, AND PROGRAM

(71) Applicant: SONY GROUP CORPORATION, TOKYO (JP)

(72) Inventors: HARUKA KUTSUKI, TOKYO (JP); YUSUKE SAKAI, TOKYO (JP); SHINICHI TERAGUCHI, TOKYO (JP)

(21) Appl. No.: 18/257,380

(22) PCT Filed: Dec. 8, 2021
(86) PCT No.: PCT/JP2021/045034

§ 371 Date: Jun. 14, 2023

(30) Foreign Application Priority Data
Dec. 22, 2020 (JP) 2020-212057

(51) Int. Cl. A61M 21/02; A61M 21/00

(52) U.S. Cl. CPC A61M 21/02; A61M 2021/0022; A61M 2021/0027; A61M 2021/005; A61M 2205/3303; A61M 2205/50; A61M 2230/06

(57) ABSTRACT

The present technology relates to an information processing device, an information processing method, and a program that enable a user to be subjectively and truly satisfied. An information providing system estimates a resonant state between a user and a constituent member other than the user in a cluster including the user on the basis of biometric data of the user, and feeds back information to the user on the basis of the resonant state. The present technology can be applied to the information providing system.

### TECHNICAL FIELD

The present technology relates to an information processing device, an information processing method, and a program, and more particularly to an information processing device, an information processing method, and a program that enable a user to be subjectively and truly satisfied.

### BACKGROUND ART

In recent years, a service such as targeting advertisement, booking, or matching utilizing “AI” by machine learning or the like has been provided (see Patent Document 1).

However, the service described above is often designed on the basis of a local indicator such as maximization of the profit of the service provider and specific customers as a value axis in capitalism. Therefore, the hyperdiversity (megadiversity) of a customer, a group, and a system including them is not necessarily maximized.

Furthermore, in particular, in the present day in which an environment recognition method has taken root widely, the user is stimulated by a superficial and shallow recognition function by an immediate interaction, intervention, rendering or the like in the above-described service, and is therefore urged to take a short-sighted action.

### CITATION LIST

### Patent Document

Patent Document 1: Japanese Patent Application Laid-Open No. 2017-207974

### SUMMARY OF THE INVENTION

### Problems to be Solved by the Invention

While being urged to take such a short-sighted action, the user perceives a manipulation or an influence by intention “not visible” from such a service or the like in latent cognition, and always feels uncomfortable in the integrated field of consciousness. As a result, the user has low “sense of initiative”, “creativity”, and “sense of connection with the world” and “unnatural feeling” in various occasions, which lead to a lack of “happiness”.

The present technology has been made in view of such a situation, and enables the user to be subjectively and truly satisfied.

### Solution to Problems

An information processing device according to one aspect of the present technology includes an information processing unit that estimates a resonant state between a user and a constituent member other than the user in a cluster including the user and on the basis of biometric data of the user, and feeds back information to the user on the basis of the resonant state.

In one aspect of the present technology, a resonant state between a user and a constituent member other than the user in a cluster including the user is estimated on the basis of biometric data of the user, and feeds back information to the user on the basis of the resonant state.

### BRIEF DESCRIPTION OF DRAWINGS

FIG. 1 is a block diagram illustrating a configuration example of an embodiment of an information providing system to which the present technology is applied.

FIG. 2 is a diagram illustrating an example of a data acquisition method.

FIG. 3 is a diagram illustrating an example of an illustration of a personal relationship network.

FIG. 4 is a diagram illustrating another example of the illustration of the personal relationship network.

FIG. 5 is a diagram illustrating examples of a feedback method.

FIG. 6 is a flowchart explaining processing of the information providing system 11 in FIG. 1.

FIG. 7 is a flowchart explaining a process of setting parameters of a cluster in step S11 of FIG. 6.

FIG. 8 is a flowchart illustrating a data acquisition process in step S12 in FIG. 6.

FIG. 9 is a flowchart explaining a resonant state estimating process in step S14 in FIG. 6.

FIG. 10 is a diagram illustrating a display example of a group chat.

FIG. 11 is a diagram illustrating a display example of a moving image by notification.

FIG. 12 is a diagram illustrating a basic display example in the group chat.

FIG. 13 is a diagram illustrating a first display example of a positive effect.

FIG. 14 is a diagram illustrating a second display example of the positive effect.

FIG. 15 is a block diagram illustrating a configuration example of a computer.

### MODE FOR CARRYING OUT THE INVENTION

Hereinafter, an embodiment for implementing the present technology will be described. The description will provide information in the following order.
1. Embodiment
2. Specific examples
3. Others

### 1. EMBODIMENT

### <Configuration Example of System>

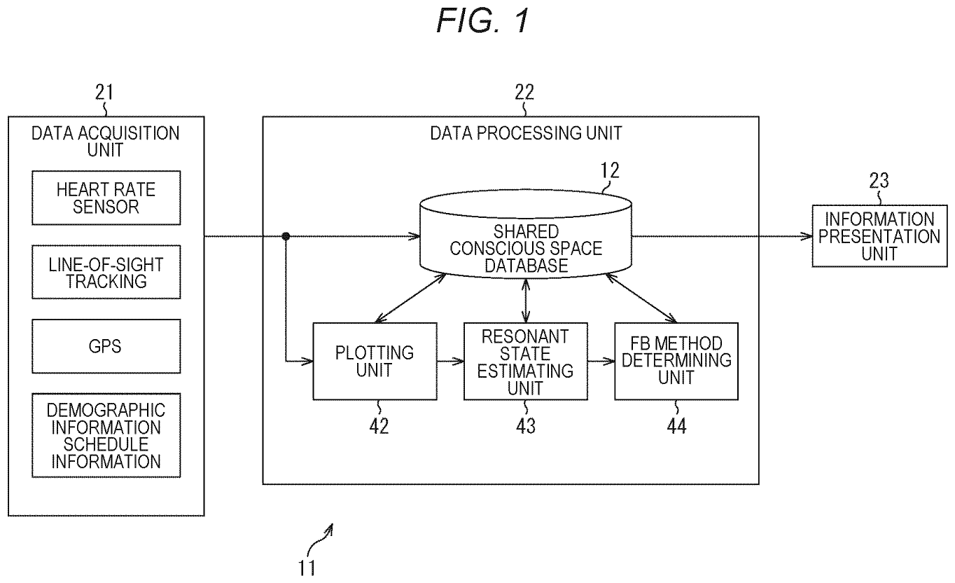

FIG. 1 is a block diagram illustrating a configuration example of an embodiment of an information providing system as an information processing system to which the present technology is applied.

An information providing system 11 in FIG. 1 is a system that performs the following three operations on a latent desire that each of a large number of users has.
(1) The information providing system 11 assumes a collective conscious space (hereinafter referred to as a shared conscious space) shared by the users, and arranges information that enables a latent desire of the users to be estimated in the shared conscious space.
(2) The information providing system 11 estimates a resonant state in consciousness and interest among the users in the shared conscious space.
(3) The information providing system 11 performs sensory information presentation to the users as recommendation of information on the basis of the estimated resonant state.

Although the existence of the shared conscious space is not verified, many concepts suggesting the possibility of the shared conscious space have been proposed. Therefore, here, the shared conscious space is based on the concepts of coherence and global coherence proposed by HeartMath Institute, which are one of many proposed concepts. Coherence refers to a stable state in which the heartbeat rhythm is consistent. This coherence also has force that also affect the surroundings. It is global coherence that implements this force of coherence at the global level.

In a cluster that is a grouping of individuals, the shared conscious space is assumed as a conscious field that is a field where collective consciousness is connected. In this conscious field, an individual is connected with a cluster across time and space, and sometimes resonates with the cluster. A database for reproducing this in a pseudo manner constructed on a cloud is a shared conscious space database 12. In the shared conscious space database 12, a user is arranged in a personal relationship network based on information of friendship input by the user and another person.

The information providing system 11 includes a data acquisition unit 21, a data processing unit 22, and an information presentation unit 23.

The functional configuration illustrated in FIG. 1 is realized by executing a predetermined program by a CPU of a server, a user terminal, or the like, not illustrated.

The data acquisition unit 21 acquires two types of data including data obtained by sensing the state of the user and data explicitly input by the user, and outputs the two types of data to the data processing unit 22.

### (Data Obtained by Sensing State of User)

Examples of the data obtained by sensing the state of the user include biometric data that is known to represent one aspect of psychological information of human such as a heart rate obtained from a heart rate sensor, a biometric potential, or a tone of voice, line-of-sight information obtained by line-of-sight tracking, position information acquired by a global navigation satellite system (GNSS), and the like.

The biometric data is data acquired in real time and in a non-invasive manner, and may also be data that can estimate emotion such as an empath and tone of voice, or data such as a high frequency component/low frequency component of heart rate, myoelectric potential, or electrodermal potential.

The biometric data, the line-of-sight information, and the position information are used to identify a phenomenon, information, and an event, and to record the user's reaction when an event occurs. Furthermore, the biometric data, the line-of-sight information, and the position information are sensed without an explicit input by the user.

The psychological information estimated from the biometric data is acquired as a preference reaction to the contacted phenomenon or target. The preference reaction means a positive or negative feedback reaction. For example, in a case where the phenomenon is a single book on a bookshelf, the preference reaction is “read the title and immediately return the book to the bookshelf”, “start reading contents”, and the like. In this case, for example, the former is a negative feedback reaction, and the latter is a positive feedback reaction. The preference reaction may be obtained directly from such a specific act, or for example, may be estimated from biometric data such as heart rate fluctuation obtained when the user touches the book.

Specifically, it is known that heart rate variability and a high frequency/low frequency component of heart rate represent the state of sympathetic/parasympathetic nerve, and represent a psychological state such as human emotions including stress. Therefore, heart rate variability and a high frequency/low frequency component of heart rate are used as psychological information appearing in the biometric data in the data processing unit 22.

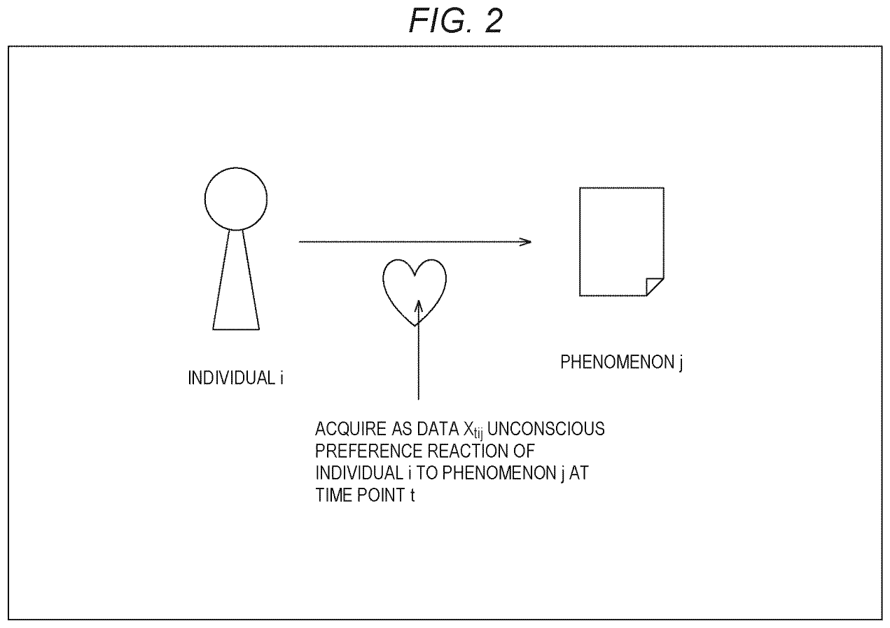

As illustrated in FIG. 2, the data acquisition unit 21 acquires as data Xₜᵢⱼ an unconscious preference reaction of an individual i to a phenomenon j at a time point t, estimated from data obtained by sensing the state of the user.

Note that the relationship between contact with a phenomenon and a physical reaction is based on the somatic marker theory. One of the differences from the prior art is that the relationship is based on the theory.

Conventionally, advertisement services and matching services for performing personalization based on machine learning have been performed. In these services, a desire of an individual has been estimated on the basis of specific information (access history or the like) obtained by explicit action of a user in a closed system such as in the Internet or in the service.

In contrast, in the present technology, future prediction that is prediction of a resonant state regarding a specific cluster (set of people, objects, and spaces) is made by sensing from an individual or a surrounding environment, and an event or an execution plan is obtained and presented to the user as a determined feedback method on the basis of the resonant state. Therefore, for example, it is possible to estimate information closer to the true desire or the latent desire of the user, such as that although the user has wanted to become good friends with a certain member, the user himself/herself couldn't conceive the possibility of becoming good friends with the member, but it is likely that the user will become good friends with the member.

### (Data Explicitly Input by User)

The data explicitly input by the user indicates demographic information and schedule information of the user used when the feedback method is determined in a later stage. The demographic information is information such as an address, an age group, a residential area, and friendship.

The demographic information is used for setting a parameter indicating a condition for configuring a cluster to which the user belongs. Note that a personal relationship network based on friendship between users registered in the shared conscious space database 12 is used to set the parameter of the cluster.

With respect to the data explicitly input by the user, the data acquisition unit 21 acquires demographic information and schedule information in response to the user's input, and outputs the demographic information and the schedule information to the data processing unit 22.

The data processing unit 22 includes the shared conscious space database 12, a plotting unit 42, a resonant state estimating unit 43, and a feedback (FB) method determining unit 44.

The plotting unit 42 plots the preference reaction data Xₜᵢⱼ on the shared conscious space database 12 every time the preference reaction data Xₜᵢⱼ of the individual i to the phenomenon j at the time point t is acquired by the data acquisition unit 21.

The plotting unit 42 sets a parameter of a cluster for configuring the cluster on the basis of the demographic information acquired by the data acquisition unit 21, and dynamically configures a cluster c from the individual i on the basis of the set parameter. The plotting unit 42 can also be referred to as a cluster configuring unit.

Furthermore, the plotting unit 42 estimates a collective level Lc of consciousness of the preference reaction in the cluster c. The level of consciousness is an average reaction level of the constituent members of the cluster excluding the user himself/herself to a phenomenon a in each cluster.

When the preference reaction data Xₜᵢⱼ is added, the resonant state estimating unit 43 compares the level Lc of consciousness in the cluster c and preference reaction data Xₜᵢⱼ of the individual that are sequentially updated. In a case where an error Lc−Xₜᵢⱼ between the both falls below a predetermined threshold, the resonant state estimating unit 43 estimates a resonant state, that is, there is resonance.

The FB method determining unit 44 determines a feedback method with reference to schedule information or the like of the constituent members in the cluster c in which there is estimated to be a resonance.

The information presentation unit 23 presents information to the user on the basis of the feedback method determined by the FB method determining unit 44. At that time, the information presentation unit 23 sensorily presents information. Sensory presentation is to present explicitly visual illustration associated with the information to be presented or to implicitly present an effect that highlights the information to be presented. Note that these presentations may be accompanied by audio, vibration, a change in color, or the like. Furthermore, an original information presentation method is not limited to visual presentation, and may be presentation by audio, vibration, a change in color, or the like.

As described above, in the information providing system 11, in the cluster based on the friendship of the user, the resonant state is estimated by oblivious sensing of biometric data, line-of-sight information, position information or the like, and feedback is performed to present information that increase happiness of the cluster on the basis of the resonant state.

Therefore, the user can increase subjective satisfaction when participating in an event related to the feedback, that is, the presented information, and the feeling of maktub, that is, “my participation to this event might be determined from the beginning”. Furthermore, the user can discover a new option without feeling strange or uncomfortable due to being forced to act by the service or system.

Note that the information providing system 11 in FIG. 1 may be configured in a server on a network, or part of the information providing system 11 such as the data acquisition unit 21 and the information presentation unit 23 may be configured in the user terminal, and the rest may be configured in the server. Note that examples of the user terminal include a smartphone, a personal computer, and the like possessed by the user.

### <Details of Data Processing>

Next, processing of the data processing unit 22 will be specifically described.

### (Data Plotting)

The plotting unit 42 plots (records) the preference reaction data Xₜᵢⱼ to the phenomenon j of the individual i at the time point t acquired by the data acquisition unit 21 on the shared conscious space database 12 as log data at the time point t. At this time, each piece of the preference reaction data Xₜᵢⱼ is data including the following items.
A time stamp of the time point t
The identifier of the individual i
The label of the contacted phenomenon j
A preference reaction vector to the phenomenon j
A membership probability vector Pic indicating the probability that the individual i belongs to the cluster c in a personal relationship network representing the friendship of the individual i

### (Setting Parameter of Cluster)

Note that before plotting data, the plotting unit 42 sets parameters of the cluster to which the user belongs. A personal relationship network based on the demographic information acquired by the data acquisition unit 21, that is, friendship between users registered in the shared conscious space database 12 is used to set the parameters of the cluster.

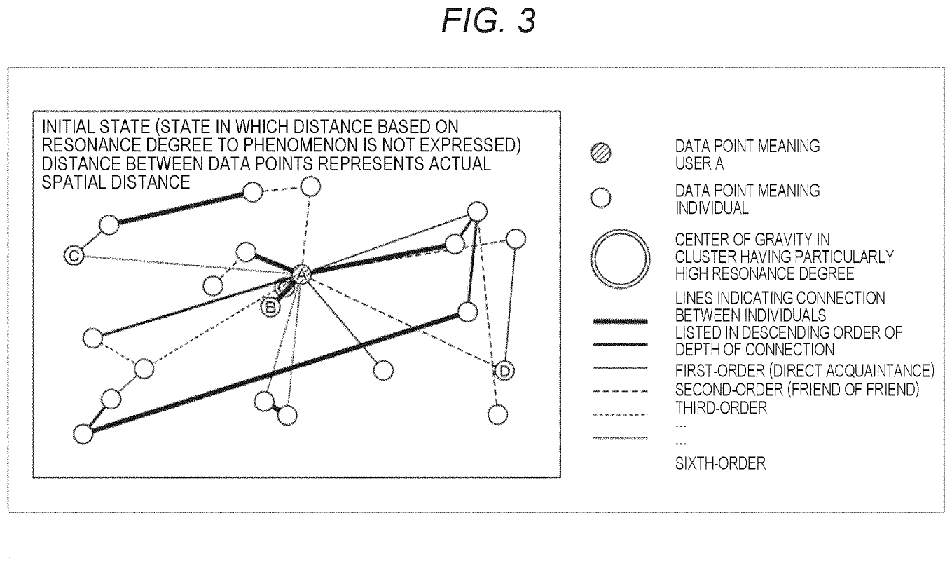

FIG. 3 is a diagram illustrating an example of illustration of a personal relationship network in a univariate data plot.

FIG. 3 illustrates illustration of the network in an initial state, that is, a state in which a distance based on a resonance degree with a phenomenon is not expressed. Therefore, the distance between data points in FIG. 3 represents an actual spatial distance viewed from a user A.

In FIG. 3, annotations for symbols are put on the right side. A circle represents a data point meaning an individual. A circle with A represents a data point meaning the user A. Circles with B to D represent data points meaning the users B to D, respectively.

A double circle represents the center of gravity in a cluster having a particularly high resonance degree, which is the degree of resonance.

Each of an extra-thick line, a thick line, a solid line, a broken line, a dotted line, and a fine dotted line represents a connection between individuals. The lines are listed in descending order of depth of connection. For example, the extra-thick line represents a first-order (direct acquaintance) connection, and a thick line represents a second-order (friend of a friend) connection. The solid line, the broken line, the dotted line, and the fine dotted line represent third-order to sixth-order connections, respectively. Note that the method of presenting connection by various lines is an example, and the presentation method is not limited to the example of FIG. 3.

For example, the data point of the user B is connected to the data point of the user A by an extra-thick line. This indicates that the user B directly knows the user A. The data point of the user B is arranged near the data point of the user A. This indicates that the user B is close to the user A also in terms of distance.

The data point of the user C is connected to the data point of the user A by a broken line. This indicates that the user C has a third-order connection with the user A. The data point of the user C is arranged away from the data point of the user A. This indicates that the user C is far from the user A in terms of distance.

The data point of the user D is connected to the data point of the user A by a fine dotted line. This indicates that the user D has a sixth-order connection with the user A. The data point of the user D is arranged away from the data point of the user A. This indicates that the user D is far from the user A in terms of distance.

In the personal relationship network configured as described above, the cluster c to which the individual i belongs is estimated from three parameters. The three parameters are the number of people in the cluster, the connection (distance) with a constituent member in the cluster when the user himself/herself is set as the center, and information indicating a specific individual who is absolutely desired to be included in the cluster.

The parameter of the number of people in the cluster is data that takes a value of two to twelve people after setting the default to four people (including the user himself/herself).

The parameter of the connection with a constituent member is the depth of friendship, and is data that takes first-order to sixth-order values after setting direct friends and acquaintances as the first-order and friends of friends as the second-order. This parameter can be arbitrarily changed by the user within a range of value.

The parameter for designating a specific individual is not set by default. By setting a parameter for designating a specific individual, a specific person can be included in a cluster, and a connection with the specific person can be manually set.

In the default state, a cluster of four second-order people including up to friends of friends is estimated. Note that as a specific example using other numerical values, for example, a cluster of four people having a first-order connection indicates a cluster of four people having a direct connection and are good friends. Furthermore, a cluster of two people having a six-order connection indicates a combination with the user himself/herself and someone who may become his/her best friend somewhere in the world.

In actual processing, estimation of a resonant state to be described later is performed in a round-robin manner, and a cluster in a highly resonant state is extracted. In a case where a parameter for designating a specific individual is set, only the remaining constituent members are estimated in consideration of the parameter. For example, in a case where there are four constituent members having a first-order connection and three people other than the user himself/herself are fixed, the cluster is uniquely determined.

### (Purpose of Cluster)

After setting the parameters of the cluster as described above, the plotting unit 42 further sets the purpose of the cluster according to the user operation. The purpose of the cluster is also one of the parameters of the cluster. The following three patterns can be set as the purposes of the cluster.
Purpose (1) wanting to actually meet a person and to spend time together, that is, including providing a real event.
Purpose (2) wanting to connect with a person online, that is, including providing an online event.
Purpose (3) wanting to touch a person or information giving a stimulus, that is, only providing information.

The purpose of the cluster set here is used for weighting at the time of determining a constraint condition and a feedback method for members to be constituent members of the cluster. The purpose of the cluster is set to (3) by default.

### (Estimation of Resonant State)

When new preference reaction data Xₜᵢⱼ is plotted by the plotting unit 42, the resonant state estimating unit 43 compares reactions Lc at the cluster c level at the same time point and time points before and after the same time point with the plotted preference reaction data Xₜᵢⱼ.

Specifically, for the cluster to be compared, in a default case, the resonant state estimating unit 43 creates all combinations from the data of the corresponding persons in a round-robin manner with the above-described default parameters (the number of constituent members is four, up to second-order connection, and the parameter for designating a specific individual is not set). Then, the resonant state estimating unit 43 sets a membership probability P_{ic}, which is a probability that the individual i belongs to the cluster c, on the basis of two points, that is, a frequency of daily communication with the constituent members and the distance of connection. The membership probability P_{ic} is set higher as the frequency of communication is higher and the connection is deeper. That is, the membership probability is a value of estimating whether or not the group is a friendly group in reality.

The resonant state estimating unit 43 creates all combinations from the data of the persons within the default conditions, and then obtains for each cluster a level Lc of consciousness, which is an average reaction level of the constituent members of the cluster excluding the individual i himself/herself to the phenomenon a in each cluster. When the value of an error E_{ci} calculated by the difference Lc−Xₜᵢⱼ between each level Lc of consciousness and the preference reaction data Xₜᵢⱼ falls below a predetermined criterion, the resonant state estimating unit 43 estimates that the cluster c and the constituent members (excluding the individual i) of the cluster c resonate with the individual i.

After the estimation result is obtained, the resonant state estimating unit 43 sorts the clusters in ascending order of the value of the error E_{ci}, that is, in descending order of the resonance degree representing the degree of the resonant state, and generates a list of the clusters having a high resonance degree. Note that, in a case where the resonance degrees are the same, sorting is performed in descending order by using the membership probability.

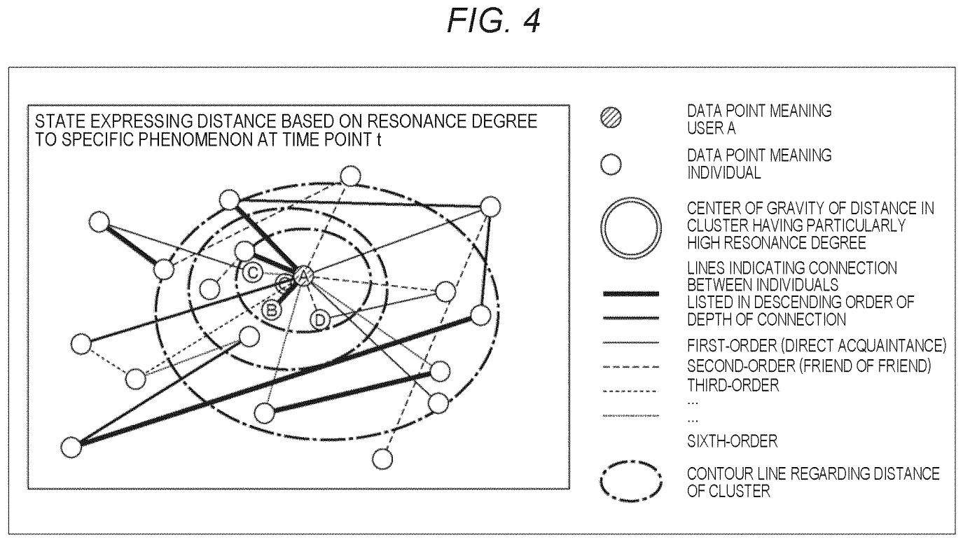

FIG. 4 is a diagram illustrating another example of illustration of the personal relationship network in a univariate data plot.

FIG. 4 illustrates illustration of the network in an initial state expressing a distance based on a preference reaction to a specific phenomenon at the time point t.

The annotations of the symbols put on the right side in FIG. 4 are similar to those in FIG. 3 except for a dashed-dotted circle. The dashed-dotted circle represents a contour line regarding the distance of the cluster. In FIG. 4, a dashed-dotted circle represents a distance based on the resonance degree with the data point of the user A, and an inner circle represents that the resonance degree is higher than that of an outer circle.

For example, data points within the innermost dashed-dotted circle at the shortest distance from the data point of the user A indicate that the resonance degrees between the user A and the users (including the users B to D) to which the data points correspond are high. For example, data points at about a medium distance from the data point of the user A, the data points being located outside the innermost dashed-dotted circle and within a second innermost dashed-dotted circle, indicate that the resonance degrees between the user A and the users to which the data points correspond are medium. Data points at a long distance from the data point of the user A, the data points being located outside the second innermost dashed-dotted circle and within the outermost circle indicate that the resonance degrees between the user A and the users to which the data points correspond are low. Moreover, data points at the longest distance from the data point of the user A, the data points being located outside the outermost circle indicate that the resonance degrees between the user A and the users to which the data points correspond are the lowest.

That is, the cluster including the users having the data points in the innermost dashed-dotted circle as the constituent members is the cluster having the highest resonance degree.

Note that, in practice, there are more user data points, and there is a plurality of clusters.

In conventional services such as targeting advertisement, booking, and matching in which personalization based on machine learning is performed, clustering based on action history and demographic information has been performed. The clustering is a process for performing the services described above.

In contrast, a network structure incorporating a personal relationship, dynamics of a cluster dynamically changing therein, and a system for comparing levels of consciousness (average turnaround reaction) at a cluster level and an individual level are novel elements of the present technology. Note that the dynamics of the cluster represents, for example, a relationship among a preference reaction associated with an event, a preference reaction of a certain person, and a preference reaction of a certain person, such as that a reaction to a certain book varies depending on the person, this person reacts to this book at this moment, who and who are in synchronization at a certain moment, and not in synchronization at the next moment.

### (Determination of Feedback Method)

After the list of clusters having a high resonance degree in which the error is less than the predetermined reference value is obtained, the FB method determining unit 44 determines the feedback method according to the situation of the cluster and the constituent members, such as a phenomenon for which the resonance degree is obtained in the cluster, the depth of the friendship which is the distance between the constituent members in the network structure of the constituent members of the cluster, and the schedule information registered by the constituent members. These pieces of information are obtained from data obtained by sensing the state of the user or data explicitly input by the user. Furthermore, as described above, the purpose of the cluster is also used for weighting at the time of determining the feedback method.

When determining the feedback method, the FB method determining unit 44 also determines which of the two types of sensory presentation methods to use. The first sensory presentation method is a method of presenting explicit visual illustration. The second sensory presentation method is a method of implicitly presenting an effect that emphasize information to be presented. That is, “implicitly” in the second sensory presentation method indicates that presentation is visually recognizable but does not have meaning as a single body, and it is conveyed that this presentation is a positive presentation when this presentation is made.

The two types of sensory presentation methods are determined according to the content of information to be presented. For example, explicit presentation is used for clear content to be presented, and implicit presentation is used for decorations of the content and user actions. Furthermore, for example, in a case where visual content related to an event such as a mountain or a book is presented, explicit sensory presentation is used, and positive presentation by an implicit effect is used for an expected action of the user such as the user making an input to the system or receiving an input from another user. Note that, in a case where three images are presented or the like, when order, effectiveness, or the like of them is indicated, explicit presentation and implicit presentation indicating additional information such as order are used in combination in some cases.

For example, at a time point when resonance is obtained in a cluster, information is provided by one of the sensory presentation methods which can give a positive impression of the resonance to the user in real time, which enables feedback that can cause the user to look at the cluster to be given to the user.

The information presentation unit 23 presents information to the user by using the feedback method and the sensory presentation method determined by the FB method determining unit 44.

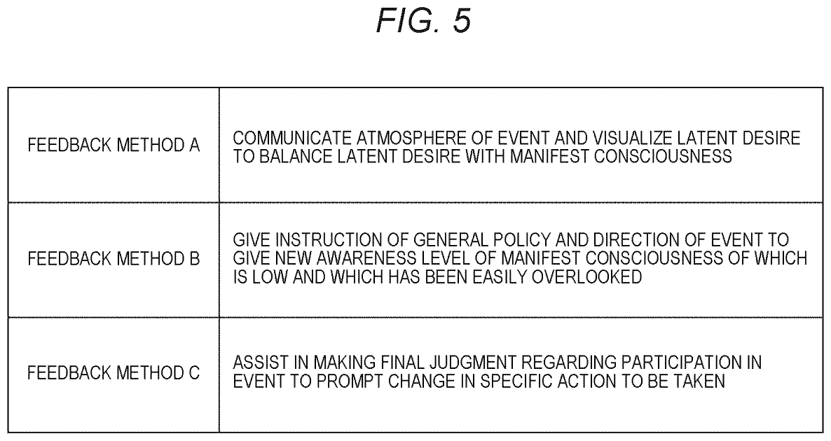

FIG. 5 is a diagram illustrating examples of the feedback method.

As described above, the FB method determining unit 44 determines the feedback method according to the situations of the cluster and the constituent members. The feedback method is not completely divided depending on the use case; however, can be divided into the following three feedback methods A to C.

The feedback method A is a method of “communicating the atmosphere of an event and visualizing a latent desire to balance the latent desire with manifest consciousness”.

Specifically, in the feedback method A, “Everyone thinks the same thing. Deep down, you want to connect with them.” is presented to convey the fact that the constituent members of the cluster are resonating (feel the same) to the user.

The feedback method B is a method of “giving an instruction of the general policy and direction of the event to give a new awareness the level of manifest consciousness of which is low and which has been easily overlooked”.

Specifically, in the feedback method B, for example, “You don't seem to be aware of this, but you seem to be curious about and interested in this. How about this?” is presented to inform the user of the new awareness or increase the opportunity to provide information regarding the new awareness.

The feedback method C is a method of “assisting in making a final judgment regarding participation in the event to prompt a change in a specific action to be taken.

Specifically, in the feedback method C, presentation is performed such that the user is encouraged when the user “wants to have an event with these people”, or the user is told that “There is an event like this. Why don't you go?”. Furthermore, the feedback method C performs presentation for prompting the user to take an action, for example, by performing preliminary preparation for actually taking an action or presenting information linked to the action such as “It is put in your calendar.” or “Why don't you go to an experience session?”.

### <Operation of System>

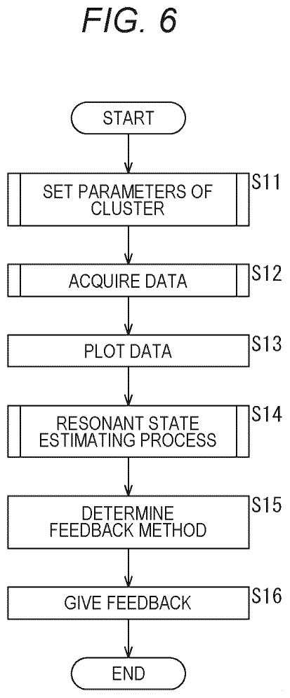

FIG. 6 is a flowchart explaining processing of the information providing system 11 in FIG. 1.

The user who uses the information providing system 11 inputs, for example, demographic information and schedule information. In response to this, the data acquisition unit 21 acquires the demographic information and the schedule information as data explicitly input by the user, and outputs the demographic information and the schedule information to the data processing unit 22.

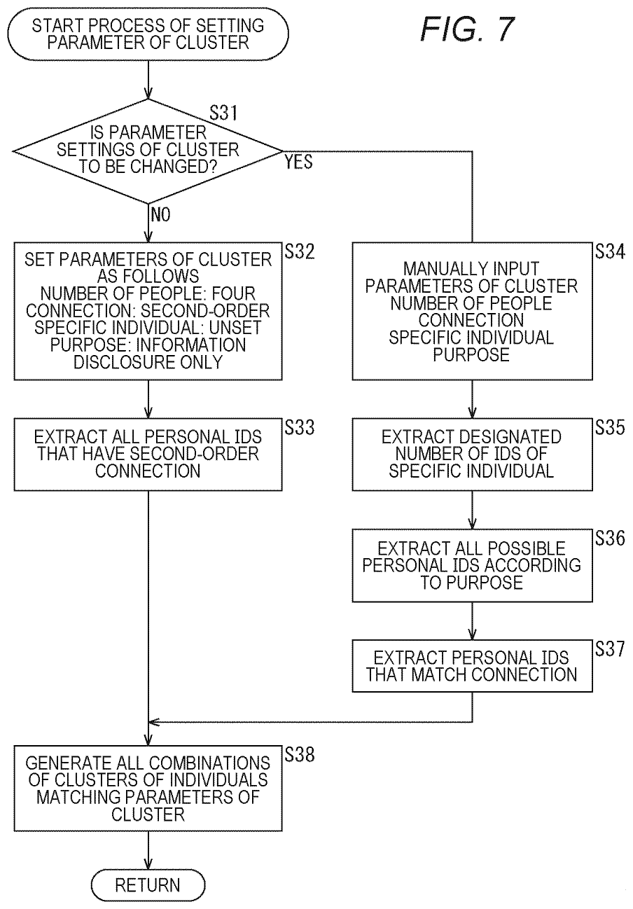

In step S11, the plotting unit 42 sets parameters of the cluster on the basis of the demographic information acquired by the data acquisition unit 21, and dynamically configures the cluster c from the individual i. The process of setting parameters of the cluster in step S11 will be described later with reference to FIG. 7.

In step S12, the data acquisition unit 21 acquires data obtained by sensing the state of the user, and outputs the data to the data processing unit 22. For example, biometric data, line-of-sight information, and position information are acquired from a sensor or the like that senses the user.

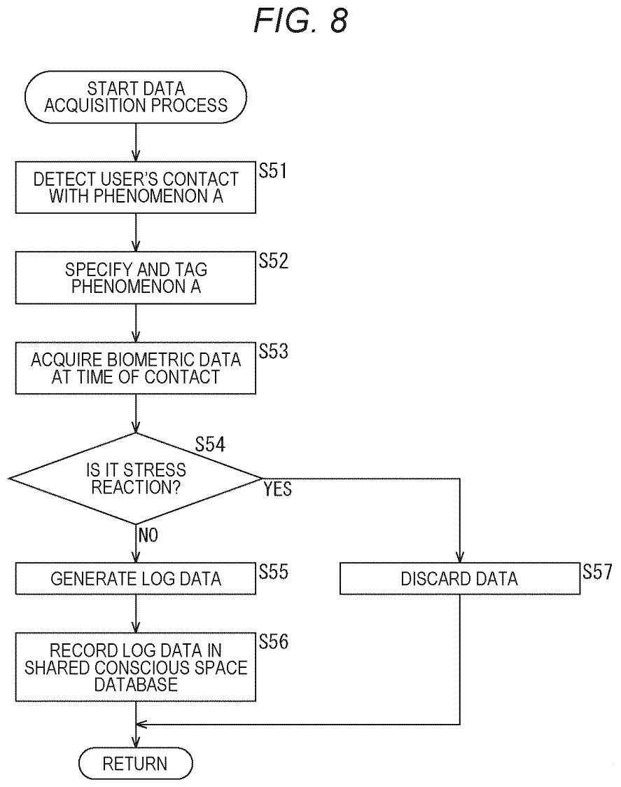

Specifically, the data acquisition unit 21 acquires data obtained by sensing the state of the user as the unconscious preference reaction data Xₜᵢⱼ of the individual i to the phenomenon j at the time point t. The process of acquiring data obtained by sensing the state of the user in step S12 will be described later with reference to FIG. 8.

In step S13, the plotting unit 42 plots the preference reaction data Xₜᵢⱼ on the shared conscious space database 12 every time the preference reaction data Xₜᵢⱼ of the individual i to the phenomenon j at the time point t is acquired by the data acquisition unit 21. At that time, the plotting unit 42 estimates a collective level Lc of consciousness of the preference reaction in the cluster c.

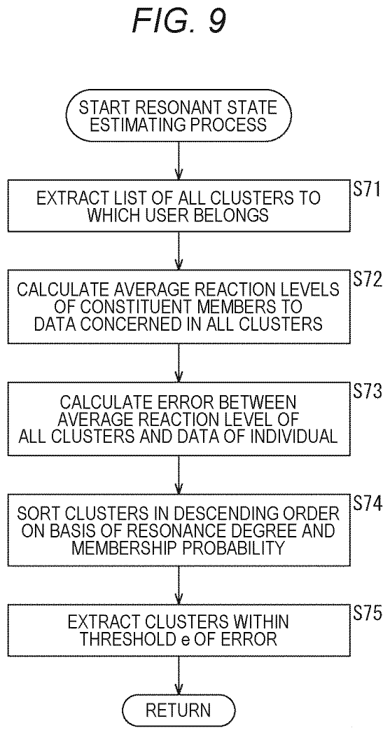

In step S14, the resonant state estimating unit 43 compares the level Lc of consciousness in the cluster c and the preference reaction data Xₜᵢⱼ of the individual that are sequentially updated at the time of adding the preference reaction data Xₜᵢⱼ, and estimates the resonant state. The resonant state estimating process in step S14 will be described later with reference to FIG. 9. In a case where the error Lc-Xₜᵢⱼ between the level Lc of consciousness and the preference reaction data Xₜᵢⱼ of the individual falls below a predetermined threshold by the process of step S14, the cluster is determined to have a high resonance degree with the individual i.

In step S15, the FB method determining unit 44 determines the feedback method according to the situations of the cluster c determined to have a high resonance degree and the situation of the constituent members in the cluster c.

For example, as in the purpose (1), in a case where the purpose is to meet directly, the FB method determining unit 44 decides to set a schedule, plan a drinking party, and make a presentation. Furthermore, as in the purpose (3), in a case where the purpose is only to get to know someone, the FB method determining unit 44 decides to present information for telling a recommended person.

In step S16, the information presentation unit 23 gives feedback and presents information to the user who is a constituent member of the cluster by the feedback method and the presentation method determined by the FB method determining unit 44.

After step S16, the processes of FIG. 6 end.

### (Process of Setting Parameter of Cluster)

FIG. 7 is a flowchart explaining a process of setting parameters of the cluster in step S11 of FIG. 6.

In step S31, the plotting unit 42 judges whether or not to change parameter settings of the cluster from the default settings on the basis of the demographic information acquired by the data acquisition unit 21. In a case where it is determined in step S31 that the parameter settings of the cluster are not changed from the default settings, the processing proceeds to step S32.

In step S32, the plotting unit 42 sets parameters of the cluster as follows. That is, the number of people: four, connection: second-order, specific individual: unset, and purpose: information disclosure only are set as parameters of the cluster.

In step S33, the plotting unit 42 extracts all the personal IDs that have a second-order connection parameter.

In a case where it is judged in step S31 that the parameter settings of the cluster are changed from the default settings, the processing proceeds to step S34.

In step S34, the plotting unit 42 sets parameters (number of people, connection, specific individual, purpose) of the cluster acquired by the data acquisition unit 21.

In step S35, the plotting unit 42 extracts a designated number of IDs of the specific individual, which is the strongest constraints among the parameters of the cluster.

In step S36, the plotting unit 42 extracts all the personal IDs with which the purpose can be achieved according to the purpose of the cluster. For example, in a case where there is a purpose of actually meeting people, if the purpose is to be performed in the near future, an individual who lives far away is not a realistic option to achieve the purpose. Therefore, the personal ID with which the purpose cannot be achieved on the basis of the residential area is excluded in the process of step S36.

In step S37, the plotting unit 42 extracts the personal IDs that match the connection parameter.

In step S38, the plotting unit 42 generates all combinations of the clusters including the personal IDs matching the parameters of the cluster, that is, the extracted personal IDs.

### (Example of Data Acquisition Process)

FIG. 8 is a flowchart illustrating a data acquisition process in step S12 in FIG. 6.

In step S51, the data acquisition unit 21 detects the user's contact with the phenomenon A. For example, it is assumed that the phenomenon A is an event of picking up a certain book.

In step S52, the data acquisition unit 21 specifies the phenomenon A by using line-of-sight information acquired from line-of-sight tracking, position information acquired from a GNSS, and the like, and performs tagging. For example, the phenomenon A is tagged with information that a book was touched, it was an AA corner, or the like.

In step S53, the data acquisition unit 21 acquires biometric data at the time of contact with the phenomenon A.

In step S54, the data acquisition unit 21 judges whether or not the user's preference reaction is a stress (negative) reaction on the basis of the acquired biometric data. In a case where it is judged in step S54 that the user's preference reaction is not a stress reaction, the processing proceeds to step S55.

In step S55, the data acquisition unit 21 generates log data by using, as the preference reaction data, the biometric data judged not to be a stress reaction, that is, judged to be a positive reaction.

In step S56, the data acquisition unit 21 registers the generated log data in the shared conscious space database 12.

Furthermore, in a case where it is judged to be a stress reaction in step S54, the processing proceeds to step S57. In step S57, the data acquisition unit 21 discards the acquired data.

After step S56 or S57, the data acquisition process ends.

### (Resonant State Estimating Process)

FIG. 9 is a flowchart explaining a resonant state estimating process in step S14 in FIG. 6.

In step S71, the resonant state estimating unit 43 extracts a list of all the clusters to which the user belongs.

In step S72, the resonant state estimating unit 43 obtains the average reaction levels of the constituent members to the phenomenon concerned in all the clusters.

In step S73, the resonant state estimating unit 43 calculates the resonance degree by calculating an error between the collective consciousness which is the average reaction level of all the clusters and data of the individual.

In step S74, the resonant state estimating unit 43 sorts the clusters in descending order of resonance degree, that is, in ascending order of error on the basis of the resonance degree and the membership probability. That is, the clusters are sorted in descending order of resonance degree. Note that, in a case where the clusters have the same resonance degrees, the clusters are sorted in descending order of membership probability.

In step S75, the resonant state estimating unit 43 extracts clusters within a threshold e of the error. The threshold e is set according to, for example, the type, purpose, or the like of the phenomenon or the event.

After step S75, the resonant state estimating process of FIG. 9 ends.

### <2. Specific Examples>

Next, information provision by the information providing system 11 configured as described above will be specifically described by using two types of use cases.

### <First Use Case>

As a first use case of the present technology, an example of connecting with a friend who is rarely seen will be described.

### (Target Person)

The targets are the user A, and a user B and a user C who are first-order friends of the user A.

### (Purpose of User)

The purpose of the user A is to be mutually and closely connected with friends who used to be good friends, whose contact information the user A knows but with whom the user A lost touch.

(Problem that User has)

Although the user A is good friends with the user B and the user C, usually, the user A always gets in touch with the user B and the user C and not vice versa, and feels that his/her affection is one-sided. The user B and the user C have affection for the user A. However, in normal times, act of getting in touch with the user A is not brought up in the consciousness of the user B and the user C. Furthermore, even if the user B and the user C recall memories of the user A at times, that does not lead to the action of getting in touch with the user A.

Therefore, the user A and the user B/user C mutually feel that they are good friends, but both of them are in a state of one-sided affection with few actions and experiences that make them to truly feel that they are good friends.

### (Approach Made by Information Providing System 11)

To be able to encourage the user B/user C to take an action of getting in touch with the user A at an appropriate timing.

### (Realization Means)

The information providing system 11 estimates a resonant state between the user B/user C and the user A, and provides a common topic such as what kind of thing they touched moves their emotions on the basis of the resonant state as needed. Therefore, it is possible to reveal synchronicity (synchrony) of the three people, and it is possible to prompt communication such that the approach described above can be made while engaging in communication. That is, the first use case is an example in which the feedback method A illustrated in FIG. 5 is used.

### (Parameters of Cluster)

Three persons including the person in question, only first-order connection, and the two constituent members are designated as specific individuals. Therefore, the cluster is uniquely determined.

### (Feedback Method)

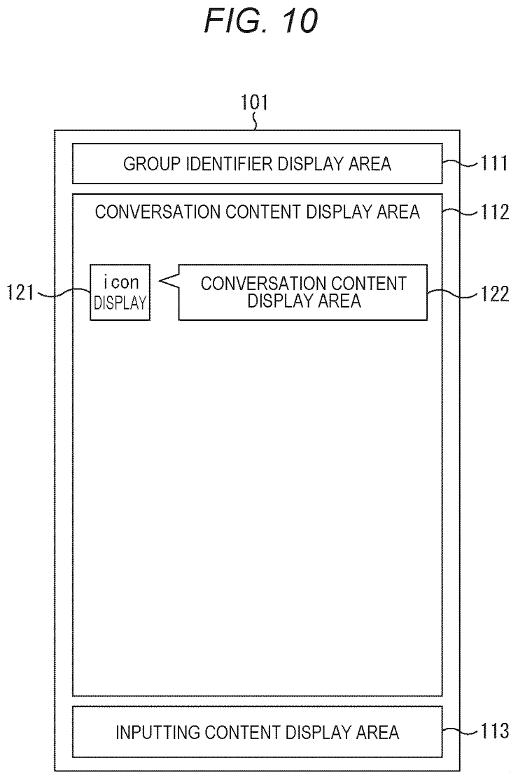

First, the FB method determining unit 44 generates a group chat of a cluster to which the three persons, that is, the user A, the user B, and the user C belong as illustrated in FIG. 10.

FIG. 10 is a diagram illustrating a display example of the group chat.

FIG. 10 illustrates a screen 101 presented to the user. On the screen 101, a group identifier display area 111, a conversation content display area 112, and an inputting content display area 113 are displayed as display of the group chat.

The group identifier display area 111 is an area in which the group name of the cluster to which the three persons, that is, the user A, the user B, and the user C belong is displayed.

In the conversation content display area 112, an icon display 121 and a conversation sentence display area 122 are displayed for each conversation sentence. The icon display 121 represents the user who has posted the conversation sentence displayed in the conversation sentence display area 122. In the conversation sentence display area 122, a conversation sentence posted by the user represented by the icon display 121 is displayed.

In the inputting content display area 113, content that is being input by the user for posting is displayed.

Note that FIG. 10 illustrates an example in which a smartphone is used as a device on which the screen 101 is presented; however, a personal computer or a television device may be used as the information presentation unit 23. The similar thing applies to the drawings illustrating the following screen examples.

Next, the data acquisition unit 21 senses the state of each constituent member of the cluster. When the resonant state estimating unit 43 estimates that a certain phenomenon resonates with the three people who are the constituent members of the cluster on the basis of the sensed data, the FB method determining unit 44 determines the feedback method so that a notification is transmitted to each constituent member.

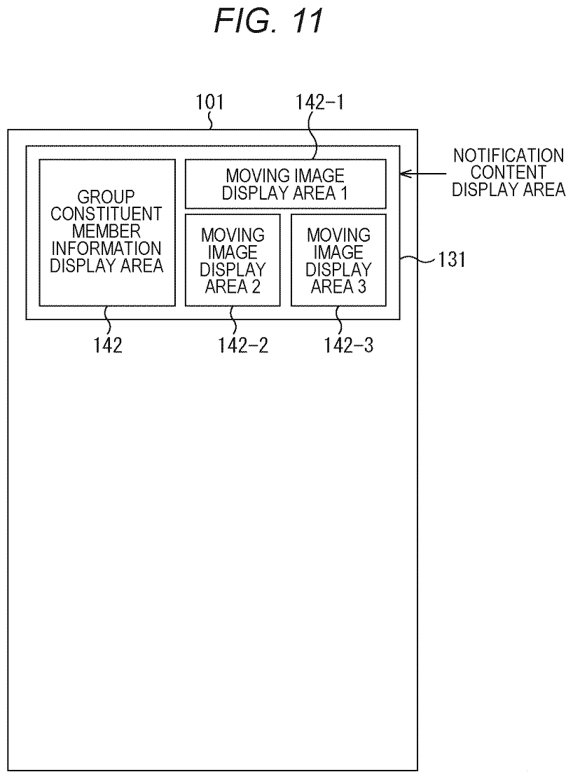

The FB method determining unit 44 determines the feedback method of presenting in substantially real time an image for illustration or an animation evoking several behaviors that are likely to be realized, the behaviors being good for the cluster, such as keeping in touch, spending time together, and crossing shoulders as illustrated in FIG. 11.

FIG. 11 is a diagram illustrating a display example of a moving image by notification.

FIG. 11 illustrates the screen 101 displayed by the information presentation unit 23. A notification content display area 131 is displayed on the screen 101. The notification content display area 131 is superimposed and displayed on part of the screen 101 in a pop-up manner when there is a notification from the information providing system 11.

The notification content display area 131 includes a group constituent member information display area 141 and moving image display areas 142-1 to 142-3.

The group constituent member information display area 141 is an area for displaying information on the constituent members of the cluster. The moving image display areas 142-1 to 142-3 are areas in which three moving images related to the resonated phenomenon are displayed.

The moving image is trimmed by cutting and extracting the size and animation according to the display area as necessary, and is displayed in the moving image display areas 142-1 to 142-3 in substantially real time.

By performing the feedback as described above, for example, the user B takes an action of getting in touch with the user A. This action is a positive and good action for the cluster. In this case, in order to indicate that the action is a good action, the FB method determining unit 44 determines an implicit feedback method by a positive effect such as a shining background of the group chat to be the presentation method.

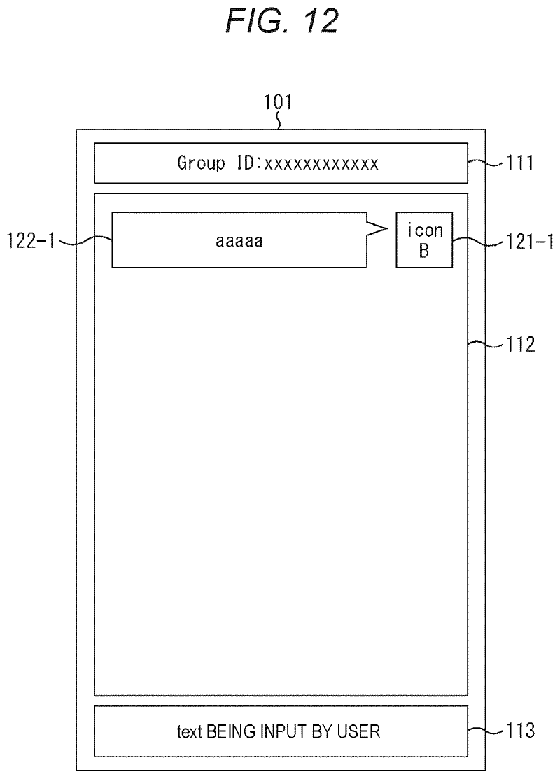

Next, a display example by a positive effect in a case where the user B gets in touch with the user A will be described with reference to FIGS. 12 to 14. Note that in FIGS. 12 to 14, portions corresponding to those in FIG. 10 are provided with the same reference signs, and description thereof will be omitted as appropriate to avoid a repetition.

FIG. 12 is a diagram illustrating a basic display example in the group chat.

In FIG. 12, “Group ID: xxxxxxxxxxxx” which is an identifier of the cluster is displayed in the group identifier display area 111. In the conversation content display area 112, an icon display 121-1 and a conversation sentence display area 122-2 are displayed. On the icon display 121-1, “Icon B”, which is an icon of the user B is displayed. In the conversation sentence display area 122-2, “aaaaa” which is a conversation sentence posted by the user B is displayed. In the inputting content display area 113, “text that is being input by the user for posting” is displayed.

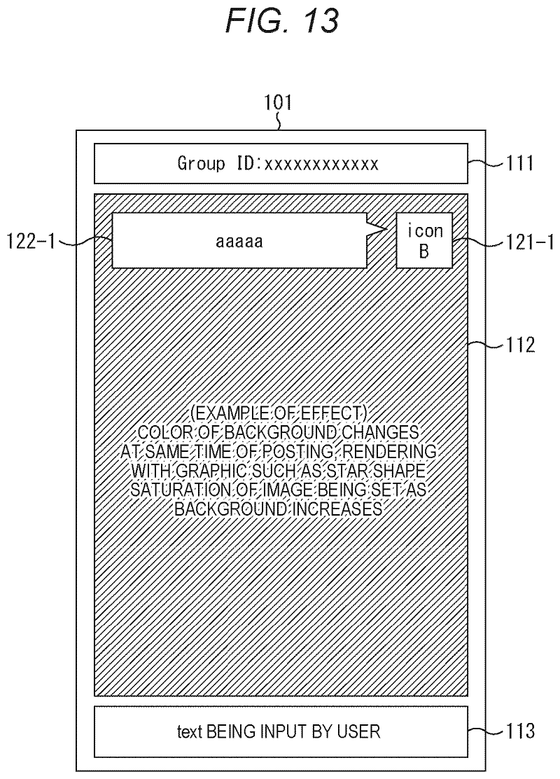

FIG. 13 is a diagram illustrating a first display example of a positive effect in the group chat.

FIG. 13 illustrates, for example, a first display example using a positive effect in a case where an action of getting in touch as one of positive actions for the cluster is sensed.

In the case of FIG. 13, hatching is applied to the background in the conversation content display area 112 as one of positive effects. This hatching indicates that a positive effect such as “the color changes”, “At the same time of posting, rendering with a graphic such as a star shape is made”, or “the saturation of the image being set increases” is used for the background.

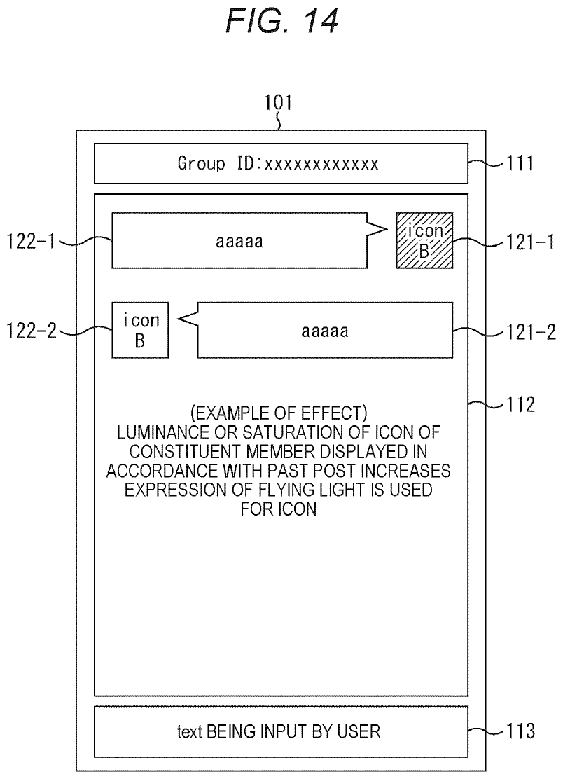

FIG. 14 is a diagram illustrating a second display example of the positive effect in the group chat.

In FIG. 14, similarly to FIG. 13, the second display example using a positive effect is illustrated.

In FIG. 14, in the conversation content display area 112, an icon display 121-2 and a conversation sentence display area 122-2 are displayed in addition to the icon display 121-1 and the conversation sentence display area 122-2. On the icon display 121-2, “Icon C”, which is an icon of the user C is displayed. In the conversation sentence display area 122-2, “aaaaa” which is a conversation sentence newly posted by the user C is displayed.

In the case of FIG. 14, as one of the positive effects, hatching is applied to the Icon display 122-1 of the user B displayed in accordance with the past post in the conversation content display area 112. This hatching indicates that a positive effect such as “changing a parameter such as saturation, luminance, or brightness” is used for the Icon display 122-1 of the user B or “superimposing an effect such as flying light” is used for the image of the Icon display 122-1 of the user B.

As described above, by using the positive effects as illustrated in FIGS. 13 and 14, it is possible to perform implicit sensory presentation for defining the action of “getting in touch” taken by the user B as a positive action. Therefore, each user can intuitively know that the action taken by the user himself/herself (the other person) was a positive action for the cluster, and can obtain subjective satisfaction.

### <Second Use Case>

As a second use case of the present technology, an example of seeking a new hobby will be described.

### (Target Person)

### User D

(Problem that User has)

The user D has some hobbies, but is getting bored these days. The user D is seeking a new hobby. Furthermore, there is no friend sharing a hobby near the user D.

### (Parameters of Cluster)

The connection is not limited to first-order and second-order connections. In the case of actually gathering, persons who are far away are excluded.

### (Feedback Method)

The information providing system 11 specifies hobby candidates that the user D himself/herself is not aware of on the basis of data obtained by sensing the state of the user D, and occasionally presents the specified hobby candidates. That is, in the second use case, the feedback method B illustrated in FIG. 5 is used.

Among the presented hobbies, the user D feeds back what the user D is interested in, such as one that the user D wishes to try, to the information providing system 11.

The data acquisition unit 21 acquires the state of the user who gives feedback on a hobby. The resonant state estimating unit 43 extracts a cluster having a high resonance degree with the state of the user that has been acquired. The FB method determining unit 44 sets a new encounter by presenting a hobby event or the like to the users of the extracted cluster, that is, the constituent members who resonate with a similar hobby. Therefore, new co-creation is generated. Note that the hobby event to be presented may be an online event or a real event.

If the user D who has participated in the presented event feeds back the evaluation to the information providing system 11, the resonant state estimating unit 43 can further extract and recommend the next cluster on the basis of the evaluation.

As described above, in the information providing system 11, a cluster can be recreated according to resonance with a hobby.

As described above, since the user D can be aware of the possibility of the hobby that the user D himself/herself was not aware of, and can actually be connected with the hobby, a cluster and other users who resonate with the hobby, the user D can obtain the subjective satisfaction.

### <3. Others>

### <Effects of Present Technology>

In the present technology, a resonant state between a user and a constituent member other than the user in a cluster including the user is estimated on the basis of biometric data of the user, and feeds back information to the user on the basis of the resonant state.

Therefore, it is possible to improve happiness for the cluster or each user in the cluster.

That is, according to the present technology, since the insensible biometric data and preference reaction of the user to a phenomenon are sensed, it is possible to estimate information closer to the true desire level and the latent desire of the user.

As a result, the user can obtain subjective satisfaction.

As described above, in the present technology, future prediction of prediction of a resonant state regarding a specific cluster (set of people, objects, and spaces) is performed by sensing from an individual or a surrounding environment, and an event or an execution plan with maximization of superdiversity from prediction results as a determination axis is obtained and presented to the user as determination of a feedback method on the basis of the resonant state. Therefore, the superdiversity of a customer, a cluster, and a system including the same can be maximized.

### <Computer Configuration Example>

The series of processes described above can be performed by hardware or can be performed by software. In a case of executing the series of processes by software, a program that constitutes the software is installed from a program recording medium into a computer built into dedicated hardware, a general-purpose personal computer, or the like.

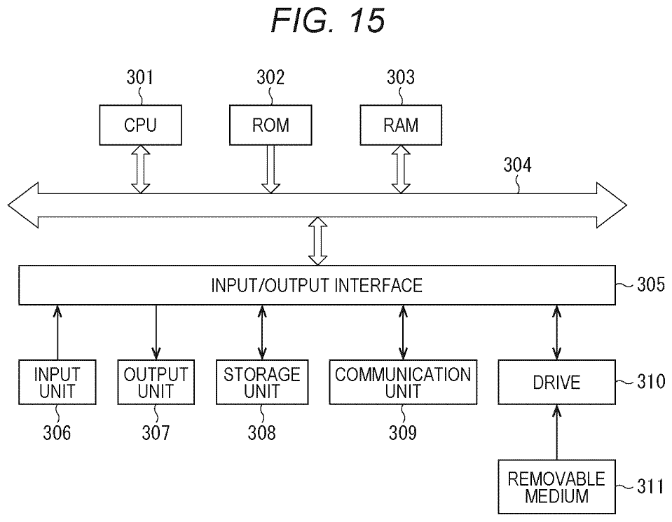

FIG. 15 is a block diagram illustrating an example of a hardware configuration of a computer that executes the series of processes described above according to a program.

A CPU 301, a read only memory (ROM) 302, and a RAM 303 are connected to one another by a bus 304.

Moreover, an input/output interface 305 is connected to the bus 304. An input unit 306 including a keyboard, a mouse, or the like, and an output unit 307 including a display, a speaker, or the like are connected to the input/output interface 305. Furthermore, a storage unit 308 including a hard disk, a nonvolatile memory, or the like, a communication unit 309 including a network interface or the like, and a drive 310 that drives a removable medium 311 are connected to the input/output interface 305.

In the computer configured as described above, the CPU 301, for example, loads the program stored in the storage unit 308 into the RAM 303 via the input/output interface 305 and the bus 304 and executes the program, and thus the above-described series of processes is performed.

The program executed by the CPU 301 is recorded, for example, on the removable medium 311 or provided via a wired or wireless transmission medium such as a local area network, the Internet, or digital broadcasting, and installed into the storage unit 308.

Note that the program executed by the computer may be a program that performs processes in chronological order according to the order described in the present Description, or may be a program that performs processes in parallel, or at necessary timing, such as when a call is made.

Note that in the present Description, a system means a set of a plurality of constituents (devices, modules (components), or the like), and it does not matter whether or not all the constituents are in the same case. Therefore, each of a plurality of devices housed in separate cases and connected via a network, and one device in which a plurality of modules is housed in one case are a system.

Furthermore, the effects described in the present Description are only examples and not limited, and other effects may also be provided.

The embodiments of the present technology are not limited to the above-described embodiments, and various modifications can be made without departing from the scope of the present technology.

For example, the present technology can adopt a configuration of cloud computing in which one function is shared and processed jointly by a plurality of devices via a network.

Furthermore, each step described in the above-described flowcharts can be executed by one device, or can be shared and executed by a plurality of devices.

Moreover, in a case where a plurality of processes is included in one step, the plurality of processes included in the one step can be shared and executed by a plurality of devices in addition to being executed by one device.

### <Examples of Configuration Combinations>

The present technology can also be configured as follows.

(1)

An information processing device including:
an information processing unit that estimates a resonant state between a user and a constituent member other than the user in a cluster including the user on the basis of biometric data of the user, and feeds back information to the user on the basis of the resonant state.
   (2)

The information processing device according to (1), in which the information processing unit includes:
a cluster configuring unit that configures a plurality of the clusters on the basis of demographic information including information indicating a personal relationship of the user, and estimates a level of consciousness of a constituent member other than the user about a phenomenon in each of the plurality of clusters;
a resonant state estimating unit that estimates the resonant state of each of the plurality of clusters by comparing the level of consciousness of each of the plurality of clusters with reaction data for the phenomenon based on the biometric data of the user; and
a feedback method determining unit that determines a feedback method to the user on the basis of the resonant state.
   (3)

The information processing device according to (2), in which the feedback method determining unit extracts the cluster the information of which is fed back on the basis of the resonant state, and determines the feedback method to the user on the basis of a situation of at least one of the cluster that has been extracted or the constituent member in the cluster.

(4)

The information processing device according to (2), in which the feedback method is any one of a method of feeding back information regarding the resonant state of the cluster to the user, a method of feeding back information that gives awareness to the user, and a method of feeding back information that prompts an action to the user.

(5)

The information processing device according to (4), in which the feedback method determining unit determines a presentation method of the information according to content of the information to be presented.

(6)

The information processing device according to (5), in which the presentation method of the information is a method of presenting an image related to the information or a method of presenting the information by using an effect for the information.

(7)

The information processing device according to (5), in which the presentation method of the information is a presentation method by display, audio, vibration, a change in color, or the like.

(8)

The information processing device according to (5) further including an information presentation unit that presents the information to the user on the basis of the feedback method and the presentation method of the information.

(9)

The information processing device according to (2), in
which the cluster configuring unit generates an illustration expressing a personal relationship of the user viewed from the user by an actual spatial distance on the basis of information indicating the personal relationship of the user.
   (10)

The information processing device according to (2), in which the cluster configuring unit sets a parameter indicating a condition for configuring the cluster in response to an operation of the user, and configures the cluster on the basis of the parameter.

(11)

The information processing device according to (10), in which the parameter includes at least one of the number of people constituting the cluster, a depth of friendship between constituent members of the cluster, a person to be included in the cluster, or a purpose of the cluster.

(12)

The information processing device according to any one of (2) to (11), in which the resonant state estimating unit extracts the cluster on the basis of a degree of the resonant state of each of the plurality of clusters and a membership probability that is a probability that the user belongs to each of the plurality of clusters.

(13)

The information processing device according to any one of (2) to (12) further including:
a data acquisition unit that acquires the biometric data from a sensor.
   (14)

The information processing device according to (13), in which the data acquisition unit specifies the phenomenon corresponding to the biometric data on the basis of position information and line-of-sight information of the user.

(15)

The information processing device according to (2), in which the resonant state estimating unit estimates the resonant state on the basis of only the biometric data judged to be a positive reaction.

(16)

The information processing device according to any one of (1) to (15), in which the biometric data includes at least one of a heart rate, a biometric potential, or a tone of voice.

(17)

An information processing method including:
causing an information processing device
to estimate a resonant state between a user and a constituent member other than the user in a cluster including the user on the basis of biometric data of the user, and to feed back information to the user on the basis of the resonant state.
   (18)

A program causing a computer to function as
an information processing unit that estimates a resonant state between a user and a constituent member other than the user in a cluster including the user on the basis of biometric data of the user, and feeds back information to the user on the basis of the resonant state.

### REFERENCE SIGNS LIST

11 Information providing system
12 Shared conscious space database
21 Data acquisition unit
22 Data processing unit
23 Information presentation unit
42 Plotting unit
43 Resonant state estimating unit
44 FB method determining unit

1. An information processing device comprising:
an information processing unit that estimates a resonant state between a user and a constituent member other than the user in a cluster including the user on a basis of biometric data of the user, and feeds back information to the user on a basis of the resonant state.

2. The information processing device according to claim 1, wherein the information processing unit further includes:
a cluster configuring unit that estimates a level of consciousness of a constituent member other than the user about a phenomenon in each of the cluster on a basis of demographic information including information indicating a personal relationship of the user;
a resonant state estimating unit that estimates the resonant state of each of the cluster by comparing the level of consciousness of each of the cluster with reaction data for the phenomenon based on the biometric data of the user; and
a feedback method determining unit that feeds back information to the user on a basis of the resonant state.

3. The information processing device according to claim 2, wherein the feedback method determining unit extracts the cluster the information of which is fed back on a basis of the resonant state, and determines the feedback method to the user on a basis of a situation of at least one of the cluster that has been extracted or the constituent member in the cluster.

4. The information processing device according to claim 2, wherein the feedback method is any one of a method of feeding back information regarding the resonant state of the cluster to the user, a method of feeding back information that gives awareness to the user, and a method of feeding back information that prompts an action to the user.

5. The information processing device according to claim 4, wherein the feedback method determining unit determines a presentation method of the information according to content of the information to be presented.

6. The information processing device according to claim 5, wherein the presentation method of the information is a method of presenting an image related to the information or a method of presenting the information by using an effect for the information.

7. The information processing device according to claim 5, wherein the presentation method of the information is a presentation method by display, audio, vibration, a change in color, or the like.

8. The information processing device according to claim 5 further comprising an information presentation unit that presents the information to the user on a basis of the feedback method and the presentation method of the information.

9. The information processing device according to claim 2, wherein the cluster configuring unit generates an illustration expressing a personal relationship of the user viewed from the user by an actual spatial distance on a basis of information indicating the personal relationship of the user.

10. The information processing device according to claim 2, wherein the cluster configuring unit sets a parameter indicating a condition for configuring the cluster in response to an operation of the user, and configures the cluster on a basis of the parameter.

11. The information processing device according to claim 10, wherein the parameter includes at least one of a number of people constituting the cluster, a depth of friendship between constituent members of the cluster, a person to be included in the cluster, or a purpose of the cluster.

12. The information processing device according to claim 2, wherein the resonant state estimating unit extracts the cluster on a basis of a degree of the resonant state of each of the cluster and a membership probability that is a probability that the user belongs to each of the cluster.

13. The information processing device according to claim 2 further comprising
a data acquisition unit that acquires the biometric data from a sensor.

14. The information processing device according to claim 13, wherein the data acquisition unit specifies the phenomenon corresponding to the biometric data on a basis of position information and line-of-sight information of the user.

15. The information processing device according to claim 2, wherein the resonant state estimating unit estimates the resonant state on a basis of only the biometric data judged to be a positive reaction.

16. The information processing device according to claim 1, wherein the biometric data includes at least one of a heart rate, a biometric potential, or a tone of voice.

17. An information processing method comprising:
causing an information processing device
to estimate a resonant state between a user and a constituent member other than the user in a cluster including the user on a basis of biometric data of the user, and to feed back information to the user on a basis of the resonant state.

18. A program causing a computer to function as
an information processing unit that estimates a resonant state between a user and a constituent member other than the user in a cluster including the user on a basis of biometric data of the user, and feeds back information to the user on a basis of the resonant state.
